(19) United States
(12) Patent Application Publication
(10) Pub. No.: US 2016/0235589 A1
(43) Pub. Date: Aug. 18, 2016

(54) POST-COITAL ABSORPTION PAD AND METHOD OF USE

(71) Applicant: Carl Rasmusen, St. George, UT (US)

(72) Inventor: Carl Rasmusen, St. George, UT (US)

(21) Appl. No.: 15/046,422

(22) Filed: Feb. 17, 2016

Related U.S. Application Data
(60) Provisional application No. 62/116,993, filed on Feb. 17, 2015.

(51) Int. Cl. A61F 13/15

(52) U.S. Cl. CPC A61F 13/15203; A61F 2013/15008; A61F 2013/8402

(57) ABSTRACT

The present disclosure is directed to absorption pads for use during sexual intercourse. In one embodiment, a post-coital absorption pad comprises an impermeable first side and at least one absorptive material on a second side.

In one embodiment, a post-coital absorption pad comprises a non-permanent adhesive-backed, impermeable first side and at least one absorptive material on a second side.

### CROSS-REFERENCE TO RELATED APPLICATIONS

This application claims the benefit of U.S. Provisional Application Ser. No. 62/116,993, filed on Feb. 17, 2015, and is incorporated herein by reference.

### TECHNICAL FIELD

The present disclosure relates to devices for use with intercourse. More specifically, the present disclosure relates to an absorption pad for post-coital discharge.

### BACKGROUND

Males and females alike may produce a certain amount of bodily discharge during sexual intercourse. This may lead to the undesired consequence of having post-coital fluids and discharge on undesirable surfaces. Due to this consequence, people have tried many methods, such as using condoms, to placing down towels to absorb the discharge. However, many couples do not need the protection of condoms or their equivalents and would prefer to do without. Further, even if using towels as an absorber, the towels often become stained and must be thrown away. Further, if towels aren't used, the bed, sheets, or other surface may become saturated with fluids and discharge. This result is especially of concern in the hospitality industry. Hotels must frequently discard sheets and towels as a result of post-coital discharge. Therefore, there remains a need for a pad that can absorb post-coital fluids and discharge and that may also be disposable. The present invention seeks to solve these and other problems.

### SUMMARY OF EXAMPLE EMBODIMENTS

The present disclosure is directed to absorption pads for use during sexual intercourse. In one embodiment, a post-coital absorption pad comprises an impermeable first side and at least one absorptive material on a second side.

In one embodiment, a post-coital absorption pad comprises a non-permanent adhesive-backed, impermeable first side and at least one absorptive material on a second side.

In another embodiment, a post-coital absorption pad comprises an impermeable first side and a plurality of removable absorptive layers on a second side.

In another embodiment, a post-coital absorption pad is tapered towards the bottom, so as to not inhibit the knees of a user during intercourse.

### BRIEF DESCRIPTION OF THE DRAWINGS

FIG. 1 is a top view of a post-coital absorption pad;

FIG. 2 is a perspective view of a post-coital absorption pad partially folded;

FIG. 3 is a perspective view of post-coital absorption pad with a plurality of absorptive layers;

FIG. 4A is a top view of a post-coital absorption pad having a drawstring;

FIG. 4B is a top perspective view of a post-coital absorption pad with the drawstring partially withdrawn; and

FIG. 4C is a side perspective view of a post-coital absorption pad with the drawstring partially withdrawn.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” does not necessarily refer to the same embodiment, although it may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various different sequences and arrangements while still falling within the scope of the present invention.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

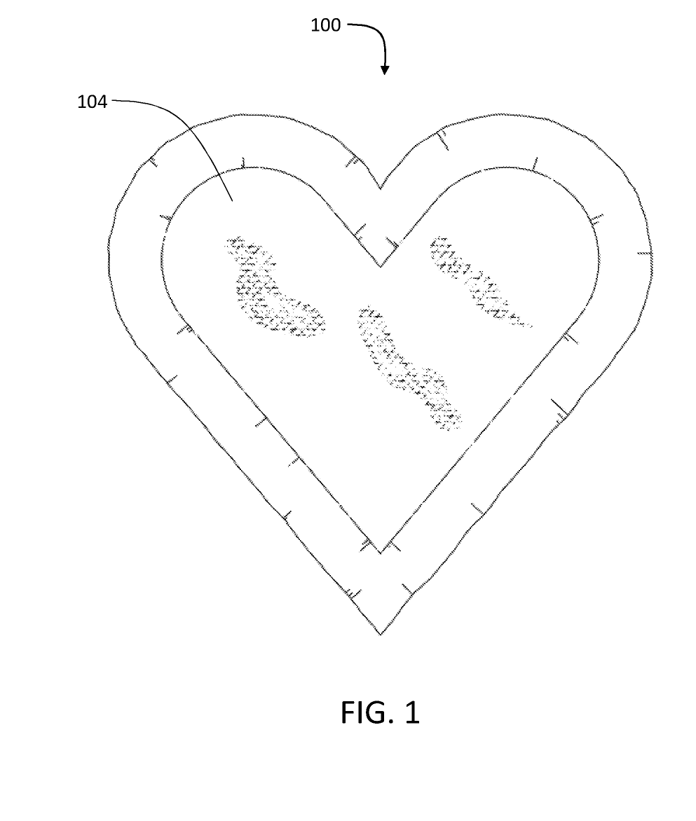

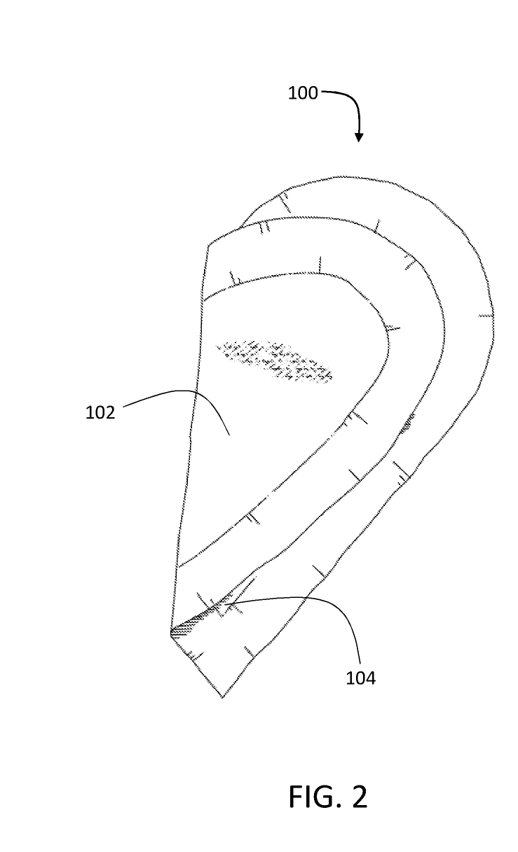

The present disclosure is directed to absorption pads for use during sexual intercourse. In one embodiment, as generally shown in FIGS. 1 and 2, a post-coital absorption pad 100 comprises an impermeable first side 102 and at least one absorptive material on a second side 104. The first side 102 may be made from various impermeable materials known in the art, such as plastic films (e.g., polyethylene) or equivalents that are impermeable to liquids, and the second side 104 may be made from absorptive materials such as cotton, bamboo, or their equivalent. The absorptive material side may further comprise absorbent gels, such as those derived from petroleum. As a non-limiting example, the post-coital absorption pad 100 may be constructed from materials used in typical sanitary napkins (e.g., Kotex® pads) used by females during menses. Similarly, the post-coital absorption pad 100 is generally the same thickness as such sanitary napkins. An example of such sanitary napkins, as well as others, is disclosed in U.S. Pat. No. 5,248,309 to Paul J. Serbiak, et al., which is incorporated herein by reference. When a couple anticipates having intercourse, they place the first side 102 on a surface (distal to the body) with the absorptive second side 104 facing toward them (proximal to the body). During and after sexual intercourse, the first side 102 of the absorption pad 100 absorbs any fluids or discharge. After completion, the couple is then free to further clean themselves, if needed, using the absorption pad 100 before discarding it. This allows a couple to have intercourse without having to worry about the clean-up and possible stains that might have otherwise existed. While FIGS. 1-3 generally illustrate the post-coital pad as heart-shaped, it will be appreciated by those in the art that any shape or form may be used.

In another embodiment, a post-coital absorption pad 100 comprises a non-permanent adhesive-backed, impermeable first side 102 and at least one absorptive material on a second side 104. The non-permanent adhesive may be located around the circumferential edges, in strips, covering the whole side, or any combination, other equivalent that prevents the pad from moving during intercourse. The non-permanent adhesive-backed, impermeable first side 102 may be made using methods and materials well-known in the art (e.g., typical pressure-sensitive adhesives made with elastomers and tackifier). The non-permanent adhesive allows the couple to enjoy intercourse without fear of the pad moving or becoming folded. For example, a couple would unfold the absorption pad 100, remove a non-permanent adhesive protective film (e.g., a thin film protective layer which protects the pad from sticking to itself and other items before use and during packaging and shipment), and then would place the pad 100, adhesive side down, on the desired location (e.g., bed, floor, table, etc.). Once intercourse has ended, the pad 100 may be gently pulled from its location and discarded.

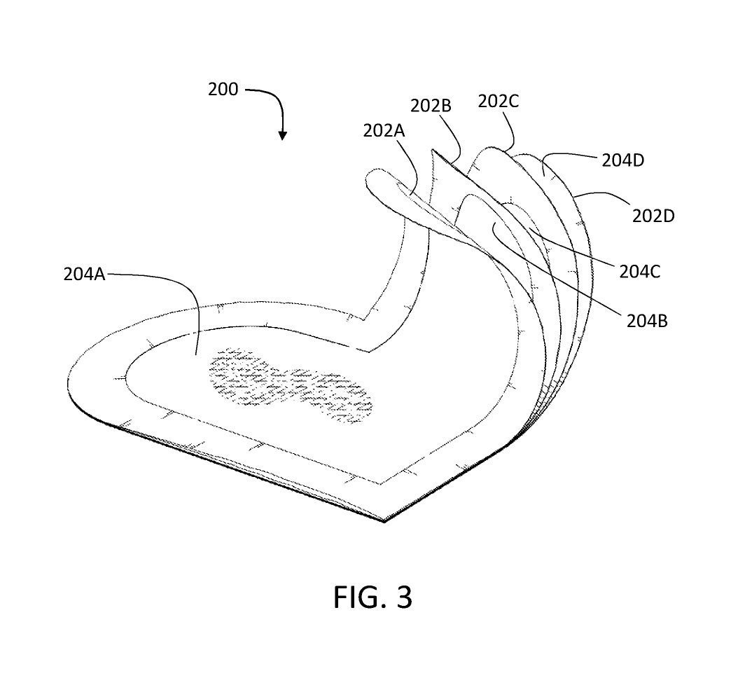

In another embodiment, as generally shown in FIG. 3, a post-coital absorption pad 200 comprises a plurality of removable layers, wherein each layer comprises an impermeable first side 202A-D and an absorptive side 204A-D. Further, impermeable first sides 202A-D comprise a non-permanent adhesive or other connecting means that keeps the various layers (each layer comprised of a first side and a second side) together until removed by a user. For example, a user would place first side 202D, which may or may not have non-permanent adhesive (because it is the bottom-most layer), on a surface for the purpose of having intercourse on the second side 204A. Once completed, a user would disconnect the top layer, 202A and 204A, by peeling it free from 204B and thereby discard it, leaving the remaining layers intact for further use. It will be appreciated that absorption pad 200 may comprise two or more layers. Further, it will be appreciated that while non-permanent adhesives may be preferred, any means to prevent movement may be used, such as hooks and loops (e.g., Velcro®) or other equivalents. In another method of use, a user may simply use each layer at a time individually. In other words, a user would peel off the top-most layer of the absorptive pad 200. The user would then place the layer with the adhesive-backed side (e.g., 202A) on a surface and firmly press to allow the layer to non-permanently adhere to the surface for use during intercourse. Once complete, the layer may be discarded.

In one embodiment, as generally shown in FIGS. 1-3, a post-coital absorption pad may be heart-shaped or otherwise tapered at one end (e.g., a triangle) so as to not inhibit the knees of a user during intercourse. In other words, in the well-known “missionary position,” the knees of the partner on top are uninhibited by the absorption pad so as to not create interference. It will be understood that the absorption pad may be a variety of sizes. In one embodiment, the absorption pad is as wide as an average female, with a corresponding length. Further, the length and width do not need to be proportionate, and any length and width sufficient to remain beneath a couple during intercourse for the collection and absorption of fluids is contemplated herein.

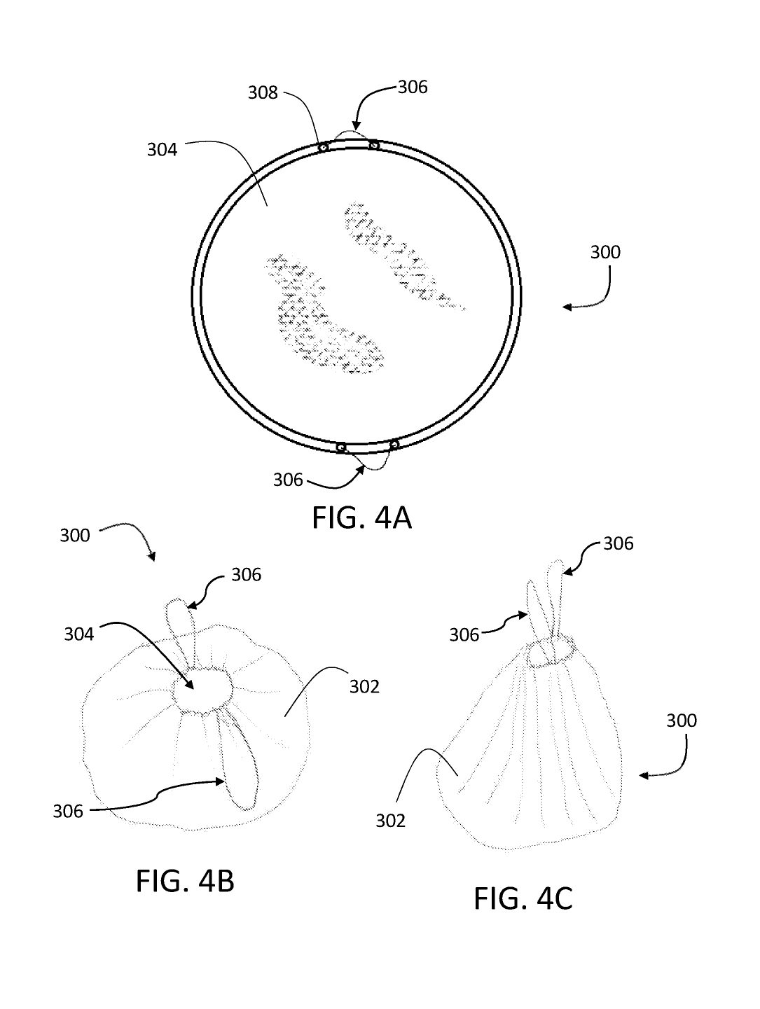

In yet another embodiment, as generally shown in FIGS. 4A-C, the circumferential edges of a post-coital absorption pad 300 may contain a drawstring 306, allowing for the absorption pad 300 to be easily closed-up, thereby retaining any fluids or discharge on the absorptive side 304 and keeping them unexposed to other surfaces. In other words, after use, a user would grab the exposed portion of the drawstring 306, pulling to withdraw the drawstring 306 through a series of apertures 308, which thereby causes the edges of the absorption pad 300 to constrict (see FIGS. 4B-C), creating a pocket for enclosing the absorption side/layer 304 of the post-coital absorption pad 300. The first side 302, which is impermeable, easily contains any fluid enclosed therein. While the drawstring is generally shown as being substantially enclosed in the circumferential edge, it will be appreciated that other common methods of utilizing a drawstring may be used, such as the over-under method of running the drawstring.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention as defined in the appended claims. Additionally, it is not intended that the scope of patent protection afforded the present invention be defined by reading into any claim a limitation found herein that does not explicitly appear in the claim itself.

What is claimed is:

1. A post-coital absorption pad, comprising:
a pad for use during intercourse, the pad having a first side and a second side, wherein the first side is impermeable to liquids and the second side is absorptive.

2. The post-coital absorption pad of claim 1, further comprising a non-permanent adhesive on at least a portion of the first side.

3. The post-coital absorption pad of claim 2, wherein the non-permanent adhesive is located on the outer circumferential edge of the first side.

4. The post-coital absorption pad of claim 3, further comprising a removable protective film over the adhesive.

5. The post-coital absorption pad of claim 1, further comprising a drawstring.

6. The post-coital absorption pad of claim 4, further comprising a drawstring.

7. A multi-layer post-coital absorption pad, comprising:
a pad for use during intercourse, the pad having a plurality of removable layers, wherein each layer comprises a first side impermeable to liquids and an absorptive second side; and
wherein at least one side of each layer comprises an adhesive.

8. The multi-layer post-coital absorption pad of claim 7, wherein at least one layer has a drawstring.

9. A method of using a post-coital absorption pad during intercourse, comprising:
placing a post-coital absorption pad on a surface;
having intercourse on the absorption pad; and
discarding the soiled absorption pad;
wherein the post-coital absorption pad comprises an impermeable first side and an absorptive second side.

10. A method of using a post-coital absorption pad during intercourse of claim 9, wherein the post-coital absorption pad further comprises a drawstring and wherein a user pulls on the string to thereby withdraw it and cause the absorption pad to constrict so as to contain any liquids.

11. A method of using a post-coital absorption pad during intercourse of claim 9, wherein the post-coital absorption pad further comprises a plurality of layers and wherein a user removes a layer either before or after intercourse.
